SEQUENCE LISTING (9 sequences)

SEQ ID NO: 1 - protein, 5 aa, Artificial Sequence
  Synthetic construct
PAPAP

SEQ ID NO: 2 - protein, 6 aa, Artificial Sequence
  Synthetic construct
LEGGGS

SEQ ID NO: 3 - protein, 4 aa, Artificial Sequence
  Synthetic construct
TGSG

SEQ ID NO: 4 - protein, 8 aa, Artificial Sequence
  Synthetic construct
GGSGGGSG

SEQ ID NO: 5 - protein, 5 aa, Artificial Sequence
  Synthetic construct
GGGGS

SEQ ID NO: 6 - protein, 4 aa, Artificial Sequence
  Synthetic construct
GGGS

SEQ ID NO: 7 - protein, 6 aa, Artificial Sequence
  Synthetic construct
GSGGGG

SEQ ID NO: 8 - protein, 5 aa, Artificial Sequence
  Synthetic construct
EAAAK

SEQ ID NO: 9 - protein, 7 aa, Artificial Sequence
  Synthetic construct
AEAAAKA

(19) United States
(12) Patent Application Publication
(10) Pub. No.: US 2023/0115540 A1
(43) Pub. Date: Apr. 13, 2023

(54) TUMOR TARGETED FLUORESCENCE GUIDANCE FOR INTRAOPERATIVE MARGIN ASSESSMENT

(71) Applicants: H. LEE MOFFITT CANCER CENTER AND RESEARCH INSTITUTE, INC., Tampa, FL (US); UNIVERSITY OF ARIZONA, Tucson, AZ (US)

(72) Inventors: David MORSE, Tampa, FL (US); Marie Catherine LEE, Tampa, FL (US); Narges TAFRESHI, Tampa, FL (US); Daruka MAHADEVAN, San Antonio, TX (US)

(21) Appl. No.: 17/909,529

(22) PCT Filed: Mar. 8, 2021
(86) PCT No.: PCT/US2021/021333

§ 371 Date: Sep. 6, 2022

Related U.S. Application Data
(60) Provisional application No. 62/986,364, filed on Mar. 6, 2020.

(51) Int. Cl. A61K 49/00; G01N 33/574; A61K 51/10; A61P 35/00; A61B 34/20; A61B 5/00

(52) U.S. Cl. CPC A61K 49/0058; G01N 33/57473; A61K 49/0034; A61K 51/1048; A61P 35/00; A61B 34/20; A61B 5/0071; A61B 5/0091; A61B 2034/2055; A61B 2017/008

(57) ABSTRACT

Disclosed are novel detectable imaging agents that can bind to a novel biomarker for cancer and methods of their use in the resection of a tumor.

### I. BACKGROUND

Over half of the 270,000 breast cancer diagnoses in the United States are for early stage disease; this is the population of women who will ultimately benefit from this research. A majority of these women will be candidates for breast conserving surgery, which consists of removal of the primary breast cancer with a surrounding margin of normal tissue. Breast conservation is associated with decreased operative risk, shorter recovery time, and decreased disruption of body image and quality of life. Furthermore, there is no difference in breast cancer mortality between lumpectomy and total mastectomy. Because of these significant advantages, breast conserving surgery continues to be the preferred operative approach for unifocal breast cancer. A key component to preservation of the breast is resection of the primary tumor with a surrounding margin of unaffected tissue; inadequate resections are associated with an increased risk in recurrence and negative effect on the patient. Current reoperative rates to obtain negative margins range between 20-50%. An estimated 35,000 reoperative procedures are performed each year after breast cancer lumpectomy due to undetectable breast cancer cells left at the time of surgery. National guidelines underscore the importance of resection to negative margins in breast conservation while clarifying the indications for additional surgery. Any repeat operation increases complication risk, cost, and duration of care. Reoperation increases risk of complications, pain, cost, and delays other breast cancer therapy, but is important in reducing the risk of recurrent cancer after treatment. The current standard of care involves a pathology exam of the removed tissue after the surgical procedure is completed to determine if the tumor was completely removed (complete resection), or if some tumor tissue was left in the patient (incomplete resection). Patients with incomplete resections will need to undergo a second surgical procedure (re-resection). Currently, surgeons are not always able to identify the extent of the disease leading to high re-resection rates where 20-50% of patients need to undergo a second surgery. Given the significant and diverse burden of reoperation, technological advances to eliminate repeat resections will have a marked impact on both the patient and the overall delivery of cancer care. What is needed is a fluorescent breast tumor targeted drug that can be imaged in real-time during breast conserving surgery (lumpectomy).

### II. SUMMARY

Disclosed are methods and compositions related to a humanized anti-CEACAM6 monoclonal antibody imaging agents.

In one aspect, disclosed herein are anti-CEACAM6 imaging agents comprising the formula Y-R, wherein Y comprises an anti-CEACAM6 binding moiety and R comprises a first detectable label; wherein the first detectable label comprises any dye (such as, for example IRdye800, AlexaFluor 790, ZW-800 (Frangioni et al), Indocyanine Green, and 50456) that emits fluorescent light that is detectable on a clinical optical NIRF imager (for example a real-time molecular fluorescence imager including but not limited to SurgVision and Fluobeam).

Also disclosed herein are anti-CEACAM6 imaging agents of any preceding aspect, wherein the anti-CEACAM6 binding moiety is comprises an antibody or CEACAM6 binding antibody fragment (such as, for example, an scFv, scFv-Fc (IgG4), F(ab′)2, or Fab′ that binds CEACAM6).

In some aspect, disclosed herein are anti-CEACAM6 imaging agents of any preceding aspect, wherein the anti-CEACAM6 binding moiety is joined to the detectable label by a NHS-ester linkage.

Also disclosed herein are anti-CEACAM6 imaging agents of any preceding aspect, wherein the anti-CEACAM6 imaging agent further comprises a second detectable label, such as, for example a radiolabel.

In one aspect, disclosed herein are the anti-CEACAM6 imaging agent of any preceding aspect for use in imaging breast cancer tissue (including, but not limited to marginal breast cancer tissue). In some aspects, the use occurs after lumpectomy and/or following systemic administration for intraoperative margin assessment.

Also disclosed herein are methods of performing optical surgical navigation comprising administering to a subject the anti-CEACAM6 imaging agent of any preceding aspect, wherein the anti-CEACAM6 imaging agent is used as an optical surgical navigation (OSN) agent administered intratumorally, peritumorally, intraperitoneally, intravenously, directly into the surgical cavity or onto the excised surgical specimen.

Also disclosed herein are methods of performing surgical removal of a tumor in a subject comprising administering to a subject the anti-CEACAM6 imaging agent of any preceding aspect; and guiding the surgical removal of the tumor using optical surgical navigation. In some aspect, the method further comprises assaying the tumor using the anti-CEACAM6 imaging agent to determine the possibility of surgical removal after administration and prior to tumor removal using optical surgical navigation. Additionally, in some aspects, disclosed herein are methods of performing surgical removal of a tumor in a subject of any preceding aspect further comprising monitoring the progress of the surgical removal.

### III. BRIEF DESCRIPTION OF THE DRAWINGS

The accompanying drawings, which are incorporated in and constitute a part of this specification illustrate several embodiments and together with the description illustrate the disclosed compositions and methods.

FIG. 1A shows mRNA expression of CEACAM6 in breast specimens.

FIG. 1B shows CEACAM6 IHC and scoring of breast TMA specimens.

FIG. 2A shows bioluminescence image of Mam-A expressing ZR-75.1 ALN metastasis. Inset shows ex vivo luminescence following resection.

FIG. 2B shows In vivo fluorescence tomographic image of ALN metastasis 24 h post administration of MamAb-800. Top inset shows ex vivo fluorescence following resection and bottom inset shows IHC of metastasis showing Mam-A expression.

FIG. 2C shows real-time fluorescence imaging during surgical removal of ALN metastasis. Arrow indicates bright MamAb-680 labeled metastasis.

FIG. 2D shows that following removal of the large metastasis, a small piece of tumor was left behind, visualized by fluorescence (arrow) and subsequently removed.

FIG. 3A shows Sensitivity of detection of cells injected into the ALN. Associated fluorescence for a range of cell numbers, 24 hours post MFP injection with MamAb-680. Inset shows fluorescence for a mouse injected with 1,000 cells. All data represent mean±SD of pixel values within the ROIs.

FIG. 3B shows a Mouse with large, —5 mm diameter, lymph node metastasis, 24 h post MFP injection with MamAb-800.

FIG. 4 shows Distance from centroid plot of fluorescence intensities across a center line through a breast cancer ALN xenograft metastasis with surrounding normal MFP tissue. Inset on right shows an overlay of ex vivo fluorescence of the ZR75.1 breast cancer xenograft metastasis on a visible light image of the tissue 24 h after intravenous administration of MamAb-800. The white line corresponds with the centroid plot. Inset on left shows an H & E section of the corresponding tissue sample with known dimensions. Dark (purple) stain indicates tumor and surrounding light (pink) stain is surrounding normal tissue.

FIG. 5 shows FGS of a SCID mouse bearing human SU.86.86 orthotopic pancreatic cancer xenograft tumor using TLR2L-800 and the clinical real-time fluorescence imaging platform (SurgVision).

FIG. 6 shows ICC of ZR-75.30, T47D and BT549 fixed cells with high, medium and low expression of CEACAM6 respectively. Blue=DAPI nuclear stain; red=CEACAM6-800 stain; green=WGA membrane stain. Left column is overlay of the three stains.

FIG. 7 shows ICC of ZR-75.30, T47D, BT549, and MCF-7 live cells with high, medium and low expression of CEACAM6 respectively. Blue=DAPI nuclear stain; red=CEACAM6-800 stain; green=WGA membrane stain. Left column is overlay of the three stains.

FIG. 8 shows Live-cell uptake of CEACAM6-800. Conjugate was added to an incubation chamber containing ZR-75.30 cells with high expression of CEACAM6 at the cell surface. The incubation chamber was mounted on an epi-fluorescence microscope and images were acquired over a 50-minute time course, and again at 90 minutes at a different field-of-view. A 400× magnification was used for the image acquisitions.

FIG. 9 shows Blocking study where a 10-fold excess of unlabeled CEACAM6 antibody was added at the same time as the CEACAM6-800. Images were acquired over a 30-minute time-course and live-cell uptake off the conjugate was significantly inhibited. 5 seconds 5 min 10 min 20 min 30 min visible light.

FIGS. 10A, 10B, 10C, and 10D show FGS of nude mice bearing MCF-7 human mammary fat pad xenograft tumors 24 h following injection with CEACAM6-800 conjugate. FIG. 10A shows reflectance fluorescence imaging prior to surgery. FIG. 10B shows real-time fluorescence imaging during surgery, and ex vivo reflectance fluorescence imaging of the excised tumor (10C) and the surviving animal (10D).

FIG. 11A shows pharacokinetics of CEACAM6-800 at a range of doses.

FIG. 11B shows tumor to adjacent tissue ratios from PK study. Note that 30 μg at 48 h post-injection provides the highest ratio.

FIG. 11C shows a BD study of the conjugate demonstrating hepatic clearance.

FIG. 11D shows the tumor specificity of CEACAM6-800 is demonstrated by blocking.

FIG. 12 shows an ex vivo image of tumor with surrounding tissue from the BD study showing fluorescence associated with tumor but not surrounding tissue.

FIG. 13A shows tumor growth delay of fluorescence guided surgery group (FGS) and controls.

FIG. 13B shows a Kaplan-Meier plot of tumor growth for the FGS group and controls.

FIG. 14 shows a representative micrographs of center sections from a resected MCF-7 xenograft tumor with surrounding tissue: H&E stained section (left), CEACAM6 IHC stained section (center), and higher magnification of a region showing the IHC stained boundary of tumor with surrounding normal tissue (right).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, pro agents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-CEACAM6 binding moiety or imaging agent is disclosed and discussed and a number of modifications that can be made to a number of molecules including the anti-CEACAM6 binding moiety or imaging agent are discussed, specifically contemplated is each and every combination and permutation of anti-CEACAM6 binding moiety or imaging agent and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The goal of this research is to develop a fluorescence molecular imaging agent that is targeted to breast tumors following systemic administration that can be used for intraoperative margin assessment.

Over half of the 270,000 breast cancer diagnoses in the United States are early stage disease. A majority of these women can be candidates for breast conserving surgery (lumpectomy), which consists of removal of the primary breast cancer with a surrounding margin of normal tissue. Removal of the tumor without removal of the breast is associated with decreased operative risk, shorter recovery time, decreased disruption of body image, and improved quality of life. Because of these significant advantages, breast conserving surgery is the preferred operative approach for early-stage, unifocal breast cancer. Inadequate resections are associated with an increased risk of recurrence and a negative effect on the patient. Current reoperative rates to obtain negative margins range between 20-50%. National practice guidelines underscore the importance of resection to negative margins in breast conservation while clarifying the indications for additional surgery. Any repeat operation increases complication risk, cost and prolongation of care. Given the significant and diverse burden of reoperation, technological advances to eliminate repeat resections can have a marked impact on both the patient and the overall delivery of cancer care. Considering the sheer number of women undergoing breast conserving surgery each year, there is potential to eliminate up to 35,000 reoperative procedures annually.

Currently, surgeons are unable to identify nonpalpable foci of disease. Intraoperative mammography, sonography, histology, cytology, and other techniques to decrease the frequency of positive margins have been studied with limited success. An ideal approach is to make residual in-breast disease visible at the time of surgery. To accomplish this, fluorescence-guided surgery (FGS) has been explored for use in lumpectomy. Unfortunately, the fluorescence imaging contrast agents tested in the clinic to date have not been specific for breast tumor relative to surrounding normal tissues, e.g. methylene blue or indocyanine green dyes, or were activatable. Activatable agents become fluorescent after cleavage by enzymes in the extracellular tumor microenvironment and are free to diffuse away from the tumor margin. Hence, these approaches have not adequately distinguished tumor margins. Herein is studied a mammaglobin-A antibody fluorescent dye conjugate to visualize breast cancer in preclinical models. However, mammaglobin-A is expressed in normal breast epithelia as well and there is concern that this could diminish the ability to detect margins, particularly in women that have breast tissue with high epithelial density. As an alternative, immunohistochemistry (IHC) staining of samples was profiled and performed from patients that were candidates for lumpectomy and have determined that CEACAM6 protein is highly and broadly expressed among candidate breast tumors, but is not expressed in surrounding normal breast tissue.

Use of a CEACAM6 specific antibody conjugated to fluorescent dye for FGS can improve primary breast cancer resection by facilitating direct visualization of in-breast disease. The end goal is to preoperatively identify patients with tumors that express CEACAM6 at core biopsy. Following systemic administration and clearance, visible fluorescence seen in the surgical cavity are then selectively resected prior to closure. To accomplish this goal, a humanized CEACAM6 specific antibody fragment can be conjugated to a near-infrared fluorescent dye and can characterize the potential for use in FGS using preclinical models of human breast cancer in immunocompromised mice and a clinical real-time fluorescence imaging platform. This novel fluorescent agent allows the detection of tumor cells in real time. Additionally, use of a CEACAM6 specific antibody conjugated to fluorescent dye for FGS can improve primary breast cancer resection by facilitating direct visualization of in-breast disease.

In one aspect, disclosed herein are any of the anti-CEACAM6 imaging agents disclosed herein for use in imaging breast cancer tissue (including, but not limited to marginal breast cancer tissue). In some aspects, the use occurs after lumpectomy and/or following systemic administration for intraoperative margin assessment.

a) Detectable Labels

In some embodiments, a detectable label (also referred to as a detectable moiety) comprises a fluorophore. As used herein, a label can include a fluorescent dye, a member of a binding pair, such as biotin/streptavidin, a metal (e.g., gold), or an epitope tag that can specifically interact with a molecule that can be detected either in vivo (e.g., after administration to a subject) and/or in vitro, such as by producing a colored substrate or fluorescence and further does not negatively impact the ability of the antibody fragment to bind to its epitope. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 7-dimethylaminocoumarin-3-carboxylic acid; 5-Carboxyfluorescein (5-FAM); 5-Carboxynapthofluorescein; 5-Carboxytetramethylrhodamine (5-TAMRA); 5-Hydroxy Tryptamine (5-HAT); 5-carboxy-X-rhodamine (5-ROX); 6-carboxy-X-rhodamine (6-ROX); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); AB Q; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 405™; Alexa Fluor 430™; Alexa Fluor 488™; Alexa Fluor 500™; Alexa Fluor 514™; Alexa Fluor 532™; Alexa Fluor 546™; Alexa Fluor 555™; Alexa Fluor 568™; Alexa Fluor 594™; Alexa Fluor 610™; Alexa Fluor 633™; Alexa Fluor 647™; Alexa Fluor 660™; Alexa Fluor 680™; Alexa Fluor 700™; Alexa Fluor 750™; Alexa Fluor 790™; Alizarin Complexon; Alizarin Red; Allophycocyanin (APC); AMC, AMCA-S; Aminomethylcoumarin (AMCA); AMCA-X; Amino actinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson −; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; cinnamic acid; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; red cyanine dyes, Cy5/Alexa 647, cyclic AMP Fluorosensor (FiCRhR); Dabcyl; Dabsyl chloride; Dansyl; Dansyl Amine; Dansyl Cadaverine; Dansyl Chloride; Dansyl DHPE; Dansyl fluoride; 4′,6-diamidino-2-phenylindole (DAPI); Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; Dronpa; bsDronpa; DsRed; DTAF; DY-630-NHS; DY-635-NHS; EBFP; ECFP; EGFP; ELF 97; EOS, Eosin; Erythrosin; Erythrosin ITC; Ethidium Bromide; Ethidium homodimer-1 (EthD-1); Euchrysin; EukoLight; Europium (111) chloride; enhanced yellow fluorescent protein (EYFP); Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; fluorescein carboxylic acid; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indocyanine Green; Indodicarbocyanine (DiD); Indotricarbocyanine (DiR); Intrawhite Cf; Li-COr dyes; IR-800 CW; IR-800 Mal; IRdye800JC-1; JO JO-1; JO-PRO-1; 50456; ZW800 and its zwitterionic drivatives; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); nitrobenzodiazolamine (NBD); NBD Amine; Nile Blue; Nile Red; NIR641, NIR664, NIT7000, and NIR782Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-CyS; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylcarboxyrhodamine; Tetraethylsulfohodamine; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; ZW-800; Sybr Green; Thiazole orange (interchelating dyes); semiconductor nanoparticles such as quantum dots; or caged fluorophore (which can be activated with light or other electromagnetic energy source), or a combination thereof.

Fluorophores emit energy throughout the visible spectrum; however, the best spectrum for in vivo imaging is in the near-infrared (NIR) region (650 nm-900 nm). Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic fluorophores with excitation and emission spectra in the NIR region. Some, such as indocyanine green (ICG) and cyanine derivatives Cy5.5 and Cy7, have been used in imaging for a relatively long time. Modern fluorophores are developed by various biotechnology companies and include: Li-COr dyes; IR-800 CW; IR-800 Mal; Alexa dyes; IRDye dyes; VivoTag dyes and HylitePlus dyes. For use in optical surgical navigation, it is not sufficient that the dye used emits in the near infrared spectrum, but needs to emit above 780 nm and can extend into the near infrared II (NIR-II) spectrum from 1000 nm to 1700 nm. An example of a detectable labels that emits between 780 nm and 1700 nm include dicyanine dye. Dicyanine dyes that are useful in this invention include IRdye800, AlexaFluor 790, ZW-800 (Frangioni et al), Indocyanine Green, 50456, and the like. Accordingly, in one aspect, disclosed herein are anti-CEACAM6 imaging agents comprising the formula Y-R, wherein Y comprises an anti-CEACAM6 binding moiety and R comprises a first detectable label; wherein the first detectable label comprises any dye (such as, for example IRdye800, AlexaFluor 790, ZW-800 (Frangioni et al), Indocyanine Green, and S0456) that emits fluorescent light that is detectable on a clinical optical NIRF imager (for example a real-time molecular fluorescence imager including but not limited to SurgVision and Fluobeam).

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT). Thus, also disclosed herein are any of the anti-CEACAM6 imaging agents disclosed herein, wherein the anti-CEACAM6 imaging agent further comprises a second detectable label, such as, for example a radiolabel.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody—antigen interactions are now commercially available. Most depend on generating an evanescent wave on a sensor surface with immobilized ligand, which allows continuous monitoring of binding to the ligand Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, 125I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner Exemplary techniques are described in Ornstein L., Disc electrophoresis—I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³⁴I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

2. Antibodies

(1) Antibodies Generally

In one aspect, the disclosed anti-CEACAM6 imaging agents comprise anti-CEACAM6 binding moiety can be an anti-CEACAM6 antibody or CEACAM6 binding antibody fragment (such as, for example, an scFv, scFv-Fc (IgG4), F(ab′)2, or Fab′ that binds CEACAM6).

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CEACAM6. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, scFv-Fc (IgG4), and the like, including hybrid fragments. Thus, fragments of the antibodies that retain the ability to bind their specific antigens are provided. For example, fragments of antibodies which maintain CEACAM6 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, scFv-Fc (IgG4), or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The delivery of the nucleic acid can be by any means, as disclosed herein, for example.

3. Linkages

In some aspect, disclosed herein are any of the anti-CEACAM6 imaging agents disclosed herein, wherein the anti-CEACAM6 binding moiety is joined to the detectable label by a NHS-ester linkage which is created using N-Hydroxysuccinimide (NHS). It is understood that other linkages can be used to join the binding moiety and detectable label. Suitable alternatives to NHS ester linkage include, but are note limited to isotheiocyante, isocyanate, sulfonyl chloride, acyl azides, anhydrides, carbonate, epoxide, difluoro, fluorophenyl ester, aldehyde, and imidoester linkages. Other suitable linkers include amino acids, peptides, nucleotides, nucleic acids, dimeric hinged Fc, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In one aspect, the NA peptide domain can be an NA4-Fc Siadel (S239D/I332E/A330L)

In one aspect, the linker may be a peptide linker. Examples of suitable peptide linkers are well known in the art, and programs to design linkers are readily available (see, e.g., Crasto et al., Protein Eng., 2000, 13(5):309-312). The peptide linker can be a restriction site linker such as the short sequence RS, or a flexible amino acid linker (e.g., comprising small, non-polar or polar amino acids). Non-limiting examples of flexible linkers include LEGGGS (SEQ ID NO: 2), TGSG (SEQ ID NO: 3), GGSGGGSG (SEQ ID NO: 4), (GGGGS)₁₋₄ (SEQ ID NO: 5), GGGS (SEQ ID NO: 6)₁₋₄, GSGGGG (SEQ ID NO: 7)₁₋₄, and (Gly)₆₋₈. Alternatively, the peptide linker can be a rigid amino acid linker. Such linkers include (EAAAK)₁₋₄ (SEQ ID NO: 8), A(EAAAK)₂₋₅A (SEQ ID NO: 9), PAPAP (SEQ ID NO: 1), and (AP)₆₋₈. The Fc domain domain can be linked to the N-terminus, the C-terminus, and/or to an internal location of the NA peptide.

In one aspect, the anti-CEACAM6 binding moiety is joined to the detectable label via click chemistry reactions. Click reactions tend to involve high-energy (“spring-loaded”) reagents with well-defined reaction coordinates, that give rise to selective bond-forming events of wide scope. Examples include, but are not limited to, nucleophilic trapping of strained-ring electrophiles (epoxide, aziridines, aziridinium ions, episulfonium ions), certain carbonyl reactivity (e.g., the reaction between aldehydes and hydrazines or hydroxylamines), and several cycloaddition reactions.

### C. METHODS OF USING THE ANTI-CEACAM IMAGING AGENTS

In one aspect, disclosed herein are methods of performing optical surgical navigation comprising administering to a subject any of the anti-CEACAM6 imaging agents disclosed herein, wherein the anti-CEACAM6 imaging agent is used as an optical surgical navigation (OSN) agent administered intratumorally, peritumorally, intraperitoneally, intravenously, directly into the surgical cavity or onto the excised surgical specimen.

Also disclosed herein are methods of performing surgical removal of a tumor in a subject comprising administering to a subject any of the anti-CEACAM6 imaging agents disclosed herein; and guiding the surgical removal of the tumor using optical surgical navigation. In some aspect, the method further comprises assaying the tumor using the anti-CEACAM6 imaging agent to determine the possibility of surgical removal after administration and prior to tumor removal using optical surgical navigation. Additionally, in some aspects, disclosed herein are methods of performing surgical removal of a tumor in a subject further comprising monitoring the progress of the surgical removal. Additionally, disclosed herein are methods of assessing the tumor margins post-excision by the pathological examination of the specimen using real-time fluorescence imaging or microscopy. The post excision examination could occur immediately within the surgical suite, or at another location.

It is understood and herein contemplated that the anti-CEACAM6 imaging agents described herein can be used in methods of treating, removing, detecting, diagnosing, prognosing, and monitoring a cancer and cancer therapies. In particular, the anti-CEACAM6 imaging agents are particularly well suited to be used in optical surgical navigation (OSN) to direct treatment and monitor the efficacy of said treatment. In some instances the anti-CEACAM6 imaging agent can be used to detect marginal breast cancer tissue following lumpectomy. Also disclosed herein methods of performing surgical removal of a tumor in a subject wherein the method is performed following systemic administration for intraoperative margin assessment

It is further understood that the disclosed methods can be used in any cancer where CEACAM6 is expressed. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. In one aspect, the cancer detected is breast cancer.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Preclinical Development of a Novel CEACAM6-Specific Antibody Conjugated to Near-Infrared Fluorescent Dye for Primary Breast Tumor Detection During Fluorescence Guided Surgery (FGS)

Breast cancer continues to be the most commonly diagnosed malignancy in American women and the second most common cause of cancer mortality in women. Nearly 270,000 new cases of invasive breast cancer and an additional 60,000 new cases of in situ carcinoma are estimated each year in the United States alone (BreastCancer.org). The primary treatment for over 80% of patients with a new breast cancer diagnosis is surgery. Surgical options for breast cancer treatment are primarily mastectomy or lumpectomy (partial mastectomy); currently, approximately 40-60% of newly-diagnosed breast cancer patients can undergo breast conserving surgery, consisting of partial mastectomy followed by radiation. A 1990 consensus statement from the National Institutes of Health advocated breast conserving surgery plus adjuvant radiation for appropriate patients presenting with stage I or stage II breast cancers.

Unfortunately, a key determinant of breast conservation is the ability to resect the primary tumor with negative margins while preserving the remaining breast. The issue of margin width is a subject of much scrutiny and debate; however, the fact remains that breast conservation is associated with a re-excision rate, despite the incorporation of multiple modalities for tumor/margin assessment and even routine additional resection. Despite the ongoing battle regarding appropriate margin width, multiple studies demonstrate the importance of clearance to surgically negative margins, as patients with positive or close margins face a significantly higher risk of local recurrence, and national consensus guidelines continue to suggest reoperation for noninvasive (DCIS) disease. The primary aim is to develop and test a novel targeted fluorescent imaging agent for improved and personalized tumor identification in primary breast cancer surgery that can be detected in real-time by intraoperative imaging.

Clinically employed intraoperative fluorescence techniques in breast cancer surgery have been limited to lymph node mapping and flap reconstruction with untargeted indocyanine green (ICG). These studies have reported high success, with detection rates and sensitivities of 98% and 95%, respectively, for lymph node staging. However, the clinical utility of untargeted near infrared fluorescent (NIRF) dyes is limited to these applications. For the successful delineation of tumors in breast conservation surgery, either targeted or activatable probes are a necessity, as untargeted constructs lack the required specificity. Preferential accumulation of 5-aminolevulinic acid (5-ALA) in breast cancer cell lines has been demonstrated in vitro and a clinical trial is being conducted in Canada to evaluate this fluorescent agent for use in discrimination of breast tumor margins. However, differential uptake in breast tumor tissue relative to surrounding normal tissue has not been reported for this agent. A group in the Netherlands has recently reported the first in-human results for use of a targeted fluorescent probe for intraoperative guidance in ovarian cancer. Multiple groups have reported the development and use of monoclonal antibody-based NIR fluorescent imaging probes for detection of primary orthotopic breast tumors in mouse models and a clinical trial for a VEGF targeting bevacizumab-IRDye800CW agent is being conducted in the Netherlands. These probes were developed by conjugating the IRDye800CW NIR fluorescent dye from LI-COR biosciences, Lincoln, Nebr. Synthetic targeted probes were developed for the cancer targets, TLR2, DOR and MC1R, which are also conjugated to the LI-COR IRDye800CW, and monoclonal antibody-based fluorescent probes conjugated to the VivoTag-680 and -800 fluorescent dyes (PerkinElmer) for the breast cancer targets mammaglobin-A (Mam-A), and carbonic anhydrases IX and XII.

a) CEACAM6 is Highly and Broadly Expressed in Patient Tumors that are Candidates for Lumpectomy, but is not Expressed in Surrounding Normal Breast Tissue.

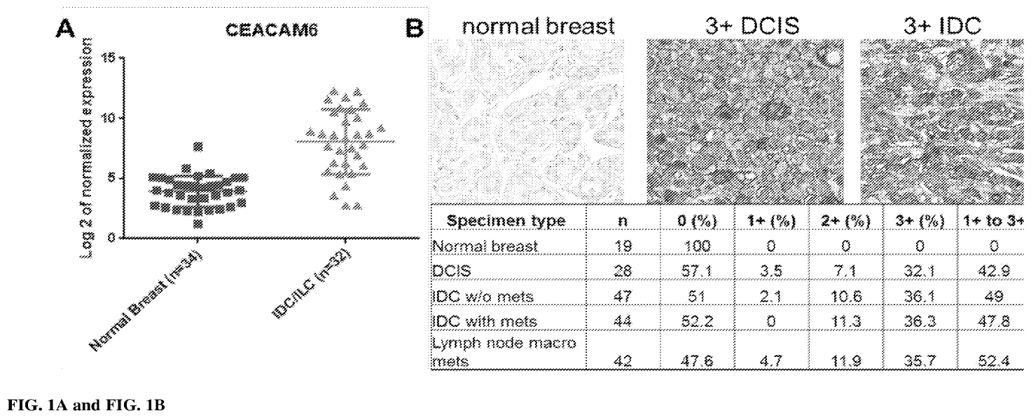

To identify targetable cell-surface markers for use in FGS, gene expression profiling of publicly available Affymetrix mRNA microarray data from patient invasive ductal and invasive lobular carcinoma tumors (n=34) and surrounding normal breast tissues (n=32) was performed. By comparing normal tissue expression levels to tumor expression levels for genes in the curated list of >5,000 genes that are potentially expressed on the cell-surface, 263 genes were identified that had higher expression in tumor relative to normal, and normal expression at or near near background levels on the microarray. From this list, 9 genes (BMPR1B, CEACAM6, CDH24, EFNA3, ERBB2, GRM8, KREMEN2, SHISA9, SLC24A2) were selected for confirmation of protein expression by IHC staining of tissue microarray sections containing 19 normal, 28 DCIS, 47 IDC without metastases, 44 IDC with metastases, and 42 lymph node macrometastases. Of the 9 proteins, only CEACAM6 (carcinoembryonic antigen-related cell adhesion molecule 6) had no expression in normal breast tissues and high and broad expression among tumor tissue types tested (FIG. 1). CEACAM6 protein is attached to the tumor cell surface via glycosylphosphatidylinositol anchor and has been reported to have expression in a number of cancer types, including breast cancer. Clearly, a single imaging agent does not work for all primary breast tumors. However, CEACAM6 can work for a large percentage of candidate tumors. Since nearly all breast tumors undergo biopsy, target expression can be determined for patients prior to scheduling surgery.

b) NIRF Antibody Conjugates have been Developed for Breast Cancer and Used for FGS in a Mouse Model.

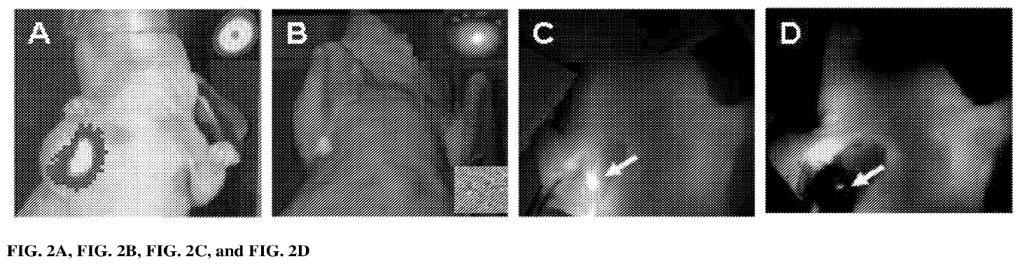

NIRF dye (VivoTag-680, PerkinElmer) was conjugated to antibodies and characterized these molecular fluorescence imaging agents for in vitro cellular uptake and in vivo tumor specificity, PK and BD. Since most clinical real-time fluorescence imaging systems were developed with the capability of imaging ICG dye, and tissue autofluorescence is reduced at these longer wavelengths, a version of the mammaglobin-A targeted probe (MamAb-800) was prepared using the VivoTag-800 dye (PerkinElmer). To demonstrate the potential for use of MamAb-800 for FGS, human ZR-75.1 axillary lymph node metastases with endogenous expression of mammaglobin-A were surgically removed using a small animal real-time fluorescence imaging system (Diagnostic Instruments) (FIG. 2).

c) Sensitivity of Margin Detection by NIRF Imaging.

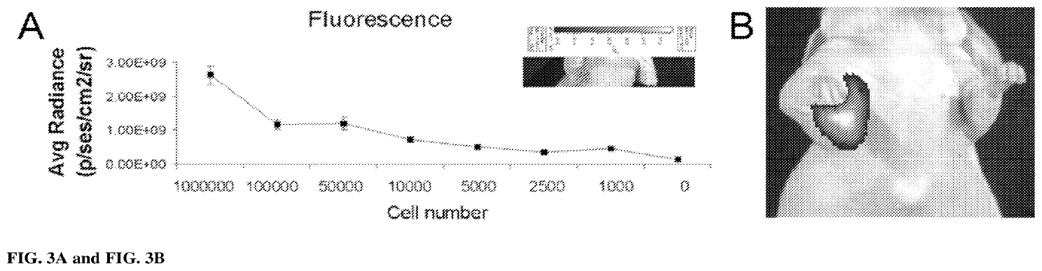

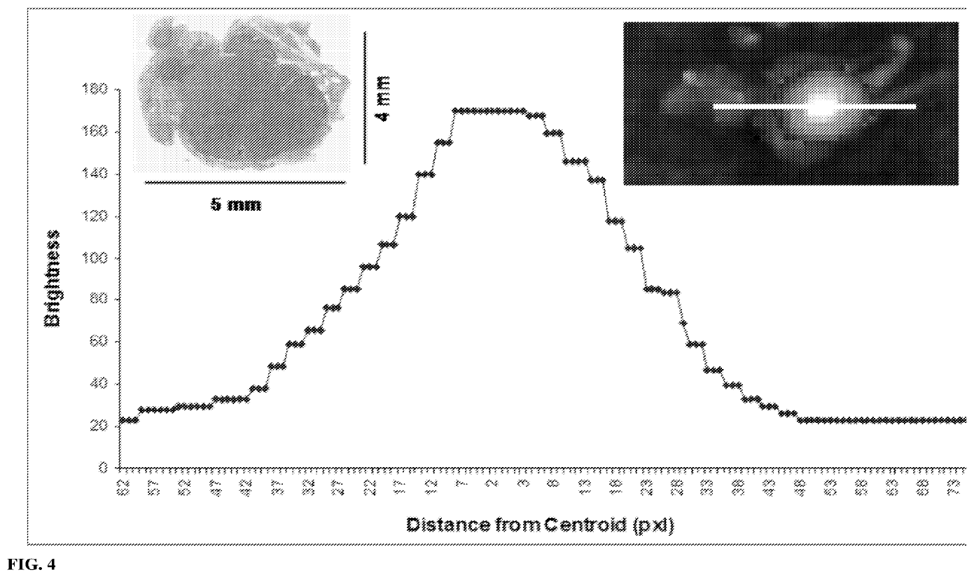

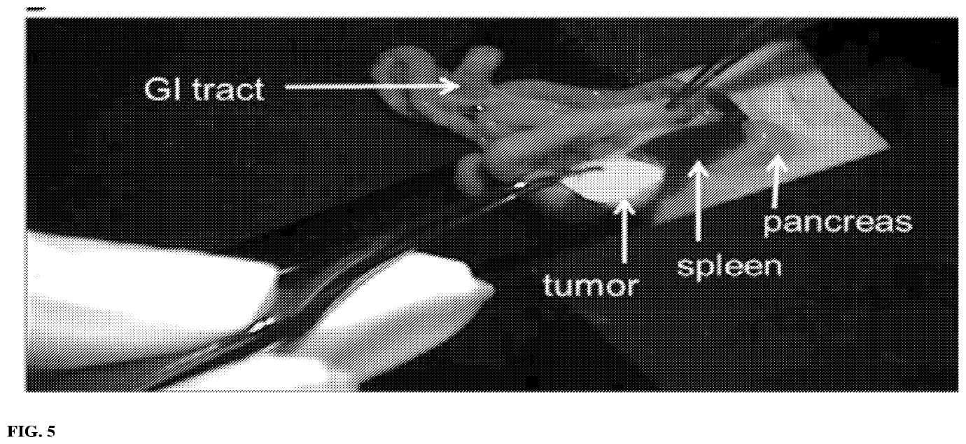

To determine the sensitivity of the agent, a range of ZR-75.1/luc cells (1000 to 1 million) were injected into the axillary lymph nodes of nude mice via ultrasound image guidance. Four hours after cell injection, MamAb-680 was injected into mammary fat pad and fluorescence images were acquired 24 h after injection (FIG. 3A). Fluorescence signals were quantified by drawing a region-of-interest (ROI) encompassing the tumor cells in the ALN. Signal intensity decreased with cell number and as few as 1,000 cells were detected. MamAb-800 had comparable results (FIG. 3B). The laboratory investigations have demonstrated that as few as 500 cells through a few (1-3) mm of tissue have been detected using the MamAb-680 probe on the IVIS 200 small animal imaging system. The closest field of view using the IVIS 200 instrument is 65×65 mm with a 2048×2048 pixels. At that magnification, each pixel represents ˜30 microns (μm), comparable to human breast tumor cellular dimensions. By generating a plot of MamAb-800 pixel fluorescence intensities through a center line of an image of a breast tumor ALN metastasis, ex vivo, and comparing the plot to an H&E image of the same tumor of known dimensions (FIG. 4), it was determined that the fluorescence extends ˜0.1 mm past the tumor edge into the margin. Based on these mouse data, a 2 mm margin of non-neoplastic tissue from fluorescent tumor cells with <1 mm error can be detected. These results from murine models confirm the sensitivity and specificity of the MamAb-680 fluorescent probe for malignant human breast tumors and support the feasibility of attaining comparable results for the planned CEACAM6 antibody fragment conjugated to NIRF dye.

d) The Clinical Real-Time Fluorescence Imaging Platform (Surgvision) for Survival FGS of Orthotopic Mouse Models of Human Cancer.

One imaging platform that can be used herein is the Surgvision clinical real-time fluorescence imaging platform. A NIRF molecular imaging agent targeted to the toll-like receptor 2 (TLR2L-800) was also developed and have used the agent for survival FGS studies using mice bearing human orthotopic xenograft tumors of pancreatic cancer (FIG. 5). Animals that received FGS lived significantly longer (some for over a year, i.e. the rest of their natural life) compared to mice in the visible-light surgery group (which lived no longer than the no-surgery controls).

e) Conjugation of Fluorescent Dye to Antibody Fragment.

For targeting, a humanized anti-CEACAM6 scFv-Fc (IgG4) antibody fragment can be used. First in human studies are currently planned to determine the safety of the unconjugated form of this antibody fragment and its potential efficacy as an anti-pancreatic cancer drug. The University of Arizona holds the intellectual property for the antibody fragment. Hence, there are no barriers toward translation of the novel antibody conjugate. The antibody fragment can be labeled and purified with the NIRF dye. The NIRF IRDye800CW (LiCor) is available with an N-hydroxysuccinimide (NHS) reactive group that couples to free amino groups on the antibody forming a stable NHS-ester conjugation. The conjugate can be purified using a Nab™ Protein L Spin Column, 0.2 mL (ThermoFisher). Protein (A280) and dye (A750) absorbance can be determined using an ND-1000 spectrophotometer (NanoDrop) and used to confirm the number of fluorophore molecules conjugated to each antibody molecule. The resulting antibody-fragment/NIRF dye conjugate can be termed CEACAM6-800.

f) In Vitro Uptake.

The human ductal carcinoma tumor cell lines BT-549, T-47D and ZR75-30 have low, medium and high expression of CEACAM6, respectively. We currently have these tumor lines, all purchased from the American Tissue Culture Collection (ATCC). Cell-surface localization of CEACAM6 can be determined. Briefly, each cell line can be fixed on slides and stained with CEACAM6-800, cell membrane stain, wheat germ agglutinin (WGA, Invitrogen) and nuclear stain (DAPI) can be included in the mounting medium (Vector Laboratories). Micrographs can be acquired using an upright epifluorescence microscope (Zeiss Imager Z2), 360-770 nm LED light source (X-Cite) and emission filters can be used for acquisitions in the blue, green and ICG wavelengths. An ORCA-Flash4.0 V3 digital CMOS camera (Hamamatsu) can be used for image acquisitions. Uptake studies can be performed, cells can be seeded on coverslips that can be transferred to an incubation chamber mounted on the fluorescence microscope. After a brief incubation with CEACAM6-800, the coverslip can be washed 3× with buffer and the growth media replaced. Images can be acquired using the ICG filter as described above over time-courses ranging from 1 minute to 2 hours. To determine specificity, the same protocol can be performed except that CEACAM6 receptor can be blocked by pre-incubation with unconjugated antibody.

g) In Vivo Dose Determination.

In in vivo fluorescence imaging studies of NIRF antibody conjugates, 50 μg of conjugate provided an optimal tumor-to-background at 24 h post-injection. Since an earlier (same-day) time-point can be optimal for clinical FGS, i.e., it is expected that a patient can be injected in the morning and have surgery in the afternoon of the same day, a dose needs to be determined where maximal tumor uptake can be distinguished from surrounding normal tissue within hours of administration. To identify the optimal dose, pairs of nude (nu/nu) mice bearing ZR75-30 mammary fat pad (MFP) xenografts (100-400 mm³ volume) can be intravenously administered a range of CEACAM6-800 concentrations, e.g., 5, 10, 20 & 40 μg in 100 μl of sterile saline, and in vivo fluorescence images can be acquired using the IVIS 200 small animal imaging system (PerkinElmer) and the ICG excitation and emission filter set over a time course of 1, 2, 4 & 24 h post-injection. The dose providing the maximum tumor to background ratio at 4 hours can be chosen for the subsequent studies. Tumor volumes can be determined by caliper measurements using the formula: volume=(length×width²)/2.

h) Blocking Study to Determine Tumor Specificity.

As described above, the optimal dose of CEACAM6-800 can be injected into one group of MFP tumor bearing animals and a second group (n=4 per group) can receive a coinjection of CEACAM6-800 with a 20-fold excess of unconjugated CEACAM6 antibody fragment. Both groups of mice can be imaged as described above at the 4 h time-point.

i) In Vivo Time-Course to Determine PK.

To determine the PK of CEACAM6-800 uptake and clearance, the associated fluorescence can be used as a surrogate for agent concentration via in vivo fluorescence imaging studies. As described above, mice (n=4) bearing MFP xenografts can be intravenously administered the optimal dose of CEACAM6-800. In vivo fluorescence images can be acquired as described above over a time course of 0, 0.75, 1.5, 3, 6, 12, 24, 48, 96, 192 and 384 h post-injection. Living Image Software (PerkinElmer) can be used to draw regions of interest (ROIs) on the acquired images over the tumor and kidneys to determine the surface radiance (photons/second/cm2/steradian).

j) Ex Vivo Study to Determine BD.

Ex vivo fluorescence imaging (IVIS 200) and epifluorescence microscopy (above) can determine tissue biodistribution and the ability to distinguish tumor margins from surrounding mammary tissue (see FIG. 4). Two groups (n=4 per group) of MFP xenograft bearing mice (described above) can be injected with the optimal dose. The first group can be imaged in vivo using the IVIS 200 at the optimal time point (determined above), and the second group can be imaged at the time-point (determined in the PK study above) that corresponds to CEACAM6-800 clearance from blood circulation Immediately following imaging, animals can be humanely euthanized and tumors and clearance organs (kidney, liver, spleen, intestines) rendered to determine tissue biodistribution. Tumors can be removed with surrounding mammary fat intact. Tumors and tissues can be gross sectioned in half and fluorescence images acquired using the IVIS 200 and SurgVision platform. H&E histological staining can be performed to determine the tumor boundary and CEACAM6 IHC can correlate fluorescence with target expression in adjacent tumor sections.

k) Survival Surgery Study.

Following injection with CEACAM6-800 agent and using the clinical real-time fluorescence imaging platform (Surgvision), surgery studies can be performed to compare the survival/recurrence rates of the following groups (n=10 per group) of ZR75-30 MFP xenografted mice: (1&2) no-surgery (with and without conjugate to control for possible direct anti-tumor effects), (3) visible light surgery and (4) fluorescence guided surgery. The surgeries can be performed using aseptic techniques and rodent survival surgery procedures. Mouse preparation can be performed in a location remote from the operating area. Mice can receive buprenorphine and ketoprofen for preemptive pain management. Mice can undergo gas anesthesia and the area of the MFP tumor can be prepared with surgical scrubs, alcohol wipes and then a betadine paint over the entire area. Following removal of the breast tumor with surrounding mammary fat, the skin layer can be closed with wound clips. Pain medication can be provided subcutaneously every 12 h for 48 h post-op then provided as needed (prn) for the next 48 h. Clips can be removed 10-14 days post-op Immediately following surgical removal, tumors can undergo ex vivo fluorescence imaging, histological staining and IHC to determine tumor margins. Mice can be observed daily for the duration of the study. Euthanasia can occur at the experimental endpoint of 180 d or at signs of protocol specified clinical endpoints, e.g. tumor burden, weight-loss, indications of lethargy, pain or distress Immediately after euthanasia, animals that reached clinical endpoints can undergo necropsy to determine the presence of cancer or infection.

l) Statistical Considerations.

The MCC Biostatistics Core performed power analyses to determine numbers for animal cohorts, i.e. to ensure that there can be sufficient power to detect differences among groups in the studies. These calculations were informed by existing results from comparable studies. When applicable, data can be analyzed for normality using the Anderson-Darling statistic. Analysis of variance (ANOVA) can be used to determine differences among groups. The Bonferroni-Holm correction for multiple testing can be used to adjust the p-values. When applicable, the Ryan-Einot-Gabriel-Welsch Multiple Range Test can be used to control the familywise error rate at α=0.05, for pairwise comparisons between the control and surgical cohorts. PK parameters can be estimated using exponential uptake and clearance fitting equations (GraphPad Prism). A multi-compartment model can also be used to estimate PK parameters. For comparison among the survival surgery study groups, Kaplan-Meier analyses can be used to evaluate time to endpoint. A non-parametric Kruskal-Wallis test can be used for quantification of IHC staining. For the ex vivo margin determination studies, 95% confidence intervals for the margin can be computed using a t-distribution. If the normality assumption is not met, an appropriate transformation, such as log-transformation, can be considered. Based on preclinical imaging data with MamAb-800, the estimated tumor margin standard deviation (SD) was about 0.5 mm Based on the data and the number of animal cases planned for the current study, the half width of the tumor margin is 0.18 mm with 95% confidence. The correlation between fluorescence resolution and CEACAM6 level of expression can be investigated using the Spearman correlation coefficient.

m) Pitfalls and Alternatives.

The antibody fragment was chosen because it is humanized and it can be appropriate for future human subjects. However, if the antibody conjugate does not perform as anticipated, other humanized anti-CEACAM6 antibodies can be used as an alternative. Similarly, IRDye800CW was chosen for conjugation because it has already been used for conjugation of targeting antibodies and peptides for a number of FGS applications and these conjugates have been tested in human subjects in Europe. However, if this NIRF dye does not perform as expected, there are a number of other ICG derivatives that can be used as alternatives. 3 breast tumor lines were chosen for the in vitro and in vivo studies. However, 6 additional breast tumor lines were identified that have CEACAM6 expression that can be used as alternatives. If the conjugate clears too slowly for a 4 h time-point, a 24 h time-point can also be used in the clinic, where the patient can receive the injection on day one, and return the next day for surgery. As an additional alternative method of delivery, the agent can be sprayed into the surgical cavity, washed and then imaged. If for some reason the SurgVision imaging platform does not perform as anticipated. The SAIL is also equipped with a clinical Fluobeam hand held real-time fluorescence imaging system (by Fluoptics) that is compatible with ICG dye and this imaging system can be used as an alternative.

### 2. Example 2: CEACAM6-Specific Antibody Conjugated to Near-Infrared Fluorescent Dye

Anti-CEACAM6 scFv-Fc (IgG4) can be conjugated to IRDye800CW (Licor) via NHS-ester linkage, purified and tested for specific uptake by human ductal breast tumor lines with a range of endogenous CEACAM6 expression levels: BT-549, T-47D and ZR75-30 (low, medium and high expression, respectively). Uptake can be determined via epifluorescence microscopy. In vivo studies can be performed using groups of nude (nu/nu) mice bearing ZR75-30 mammary fat pad (MFP) xenografts. The conjugate can be administered intratumorally, peritumorally, intraperitoneally, intravenously, directly into the surgical cavity or onto the excised surgical specimen. Blocking studies using an excess of unconjugated antibody can determine tumor specificity. An imaging time-course can determine the pharmacokinetics (PK) of uptake and clearance. Ex vivo fluorescence imaging and microscopy studies can determine tissue biodistribution (BD) and the ability to distinguish tumor margins from surrounding mammary tissue. Hematoxylin & eosin (H&E) histological staining can determine the tumor boundary and CEACAM6 IHC can correlate fluorescence with target expression in adjacent tumor sections. Using the clinical real-time fluorescence imaging platform (Surgvision), surgery studies can be performed to compare the survival/recurrence rates of the following groups of ZR75-30 xenografted mice following administration of conjugate: no-surgery (with and without conjugate to control for possible direct antitumor effects), visible light surgery and fluorescence guided surgery.

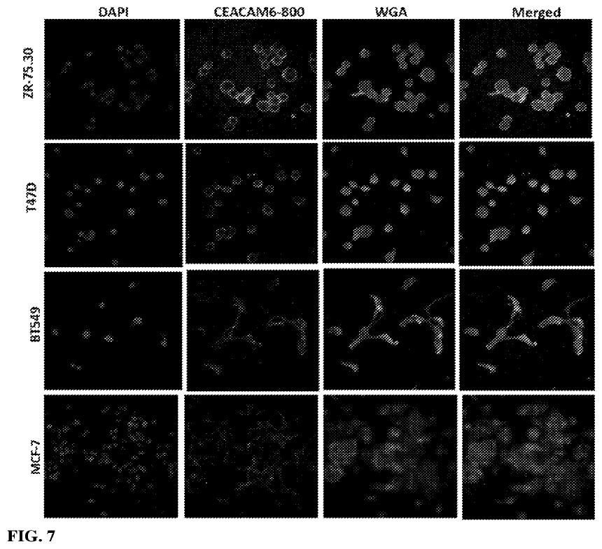

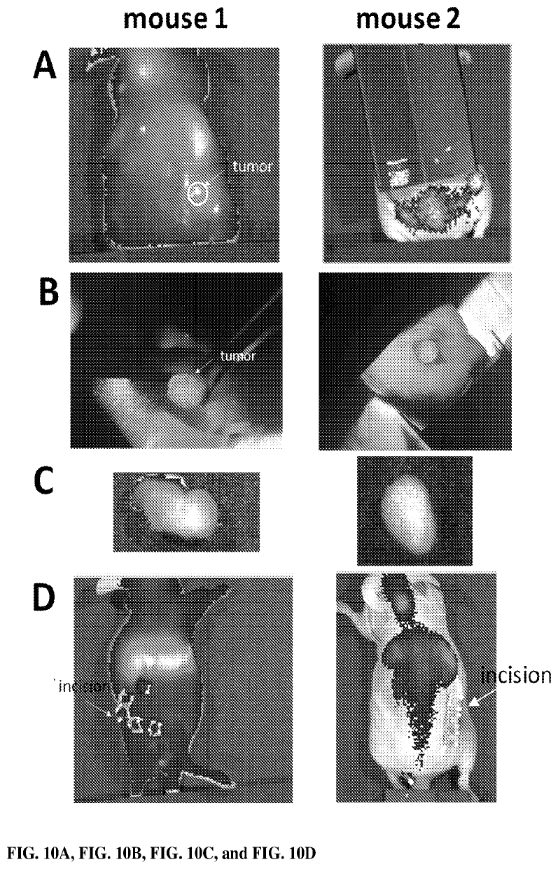

Due to slow tumor growth with the ZR75-30 xenograft model in nude mice, we chose to switch to the MCF-7 tumor model in nude mice with estrogen supplement. MCF-7 tumors have published endogenous expression of CEACAM6 at moderate levels. Immunocytochemistry (ICC) confirmed the moderate expression (FIG. 7). Unfortunately, these xenografts did not grow using estrogen supplemented in the drinking water, so estrogen pelleting was used for a pilot study of two mice (FIG. 10). As proof of principle, 30 μg CEACAM6-800 conjugate was intravenously injected into nude mice bearing MCF-7 human breast cancer mammary fat pad orthotopic xenograft tumors (n=2). Prior to FGS at 24 h, IVIS reflectance fluorescence imaging was performed, showing fluorescence in the tumor, liver, and skin, demonstrating tumor uptake and that the conjugate was still in circulation (FIG. 10A). Survival FGS was performed using the Surgvision real-time fluorescence imaging platform and high tumor fluorescence was observed relative to surrounding tissues (FIG. 10B). Post-surgery reflectance imaging was performed on the ex vivo removed tumors (FIG. 10C) and the in vivo mice (FIG. 10D), showing that tumor fluorescence was no longer observed at the site of resection.

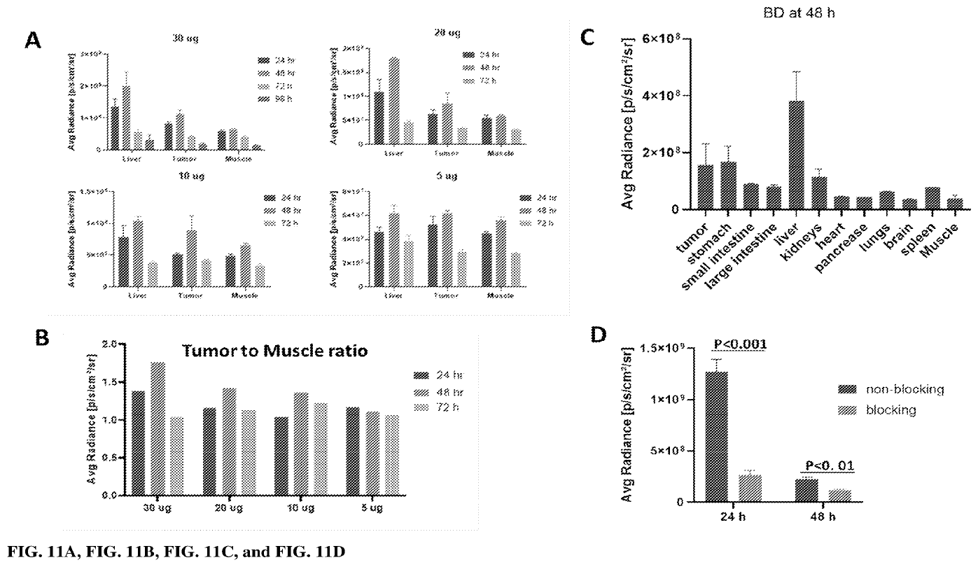

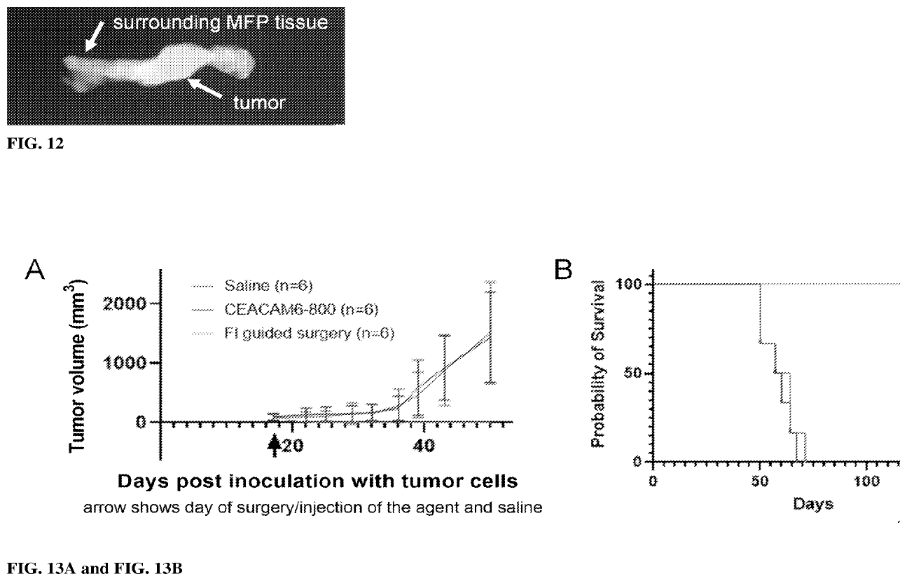

All proposed studies, goals and milestones were successfully completed. As previously reported in the 6 month and 12 month progress reports, antibody conjugation to fluorescent dye was completed and analyzed, cell lines were characterized for expression of the target, cellular uptake of the CEACAM6-800 conjugate was characterized, and FGS was performed using the conjugate. In the final 6 months, a PK time-course study was completed using a range of doses to determine the optimal dose for tumor uptake and clearance from surrounding tissues. Using nude mice bearing MCF-7 mammary fat pad tumors, fluorescence was quantified in the areas of the tumor, tissue surrounding the tumor, and liver (FIG. 11A). It was determined that the tumor to surrounding tissue ratio was highest using the 30 μg dose at 48 h post administration (FIG. 11B). A BD study was performed using the optimal dose and time point, at which tissues were resected and fluorescence images acquired ex vivo (FIG. 11C). The BD study demonstrated that clearance was primarily via the hepatic route. A blocking study was performed using the optimal dose to determine in vivo tumor specificity (FIG. 11D). Ex vivo imaging of the excised tumors with surrounding mammary fat tissue demonstrated fluorescence uptake in the tumor but not in the surrounding tissue (FIG. 12). The optimal dose and post-injection time point was used for a survival surgery study where groups of animals were administered the CEACAM6-800 conjugate or saline as a control. Animals receiving conjugate underwent survival surgery with or without real-time fluorescence imaging guidance, or no-surgery. The saline group also did not undergo surgery.

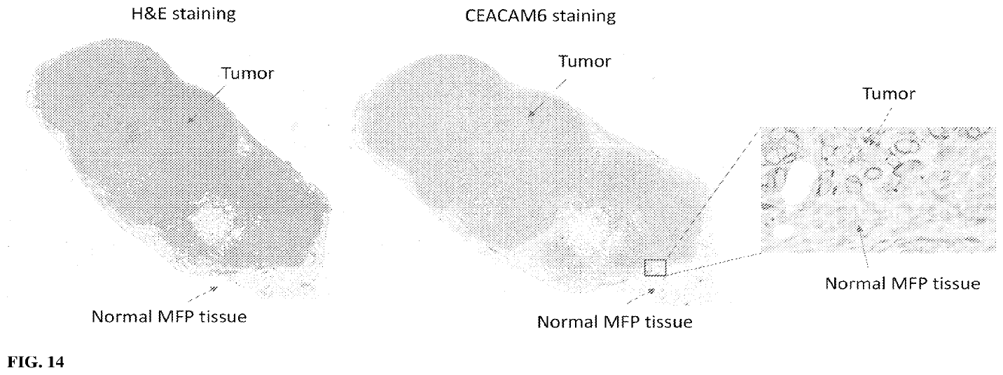

Tumor growth delay and Kaplan-Meier analyses showed that the animals that underwent surgery did not re-grow tumors and the non-surgery controls reached experimental endpoint simultaneously (FIG. 13) Animals that underwent visible light surgery also did not regrow tumors and the plot in figure three cannot be seen because it is directly under the FGS surgery group plot. This is likely due to encapsulation of the tumor which allowed for complete removal without fluorescence guidance. This is an attribute of the xenograft model that is not comparable to invasive human disease. A center section was prepared from the resected tumors and stained for histology and for CEACAM6 expression. FIG. 14 shows representative images of CEACAM6 expression in tumor but not in surrounding tissue. All margins from the tumor sections were negative.

a) Conjugation of the Antibody Fragment to Near-Infrared Fluorescent (NIRF) Dye (CEACAM6-800).

The anti-CEACAM6 scFv-Fc (IgG4) has been received from Dr. Mahadevan. The antibody fragment was conjugated to IRDye800CW (Licor) via NHS-ester linkage and purified using a spin-column for a yield 3 mg/mL via absorbance spectrometry. The conjugate was termed “CEACAM6-800”.

b) Immunocytochemistry (ICC) of Fixed Cells.

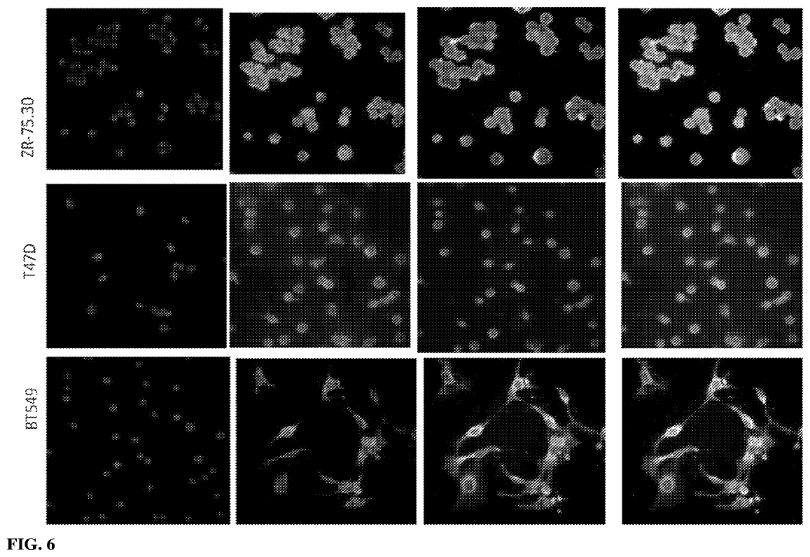

ICC was performed on fixed ZR-75.30 cells (high CEACAM6 expression), T47D cells (medium CEACAM6 expression), and BT549 cells (low CEACAM6 expression). Cells were fixed with cold methanol:acetone solution and stained with blue nuclear stain (DAPI), red CEACAM6-800 (1:30 dilution), and green membrane stain (WGA) and CEACAM6-800 (1:30 dilution). Fluorescence micrographs were acquired at 400× magnification using a confocal fluorescence microscope. See FIG. 6. The panel on the far left is an overlay of the three stains. Note the cell-surface localization of CEACAM6 (red) and the co-localization with green plasma membrane in the panels on the far right (yellow).

c) Immunocytochemistry (ICC) of Live Cells.

ICC was performed on live ZR-75.30 cells (high CEACAM6 expression), T47D cells (medium CEACAM6 expression), and BT549 cells (low CEACAM6 expression). Live cells were stained with DAPI, CEACAM6-800 and WGA for 10 minutes at 4° C. Following labeling, cells were fixed with cold methanol:acetone solution and fluorescence micrographs acquired at 400× magnification using a confocal fluorescence microscope. See FIG. 7.

d) Live-Cell Uptake Study.

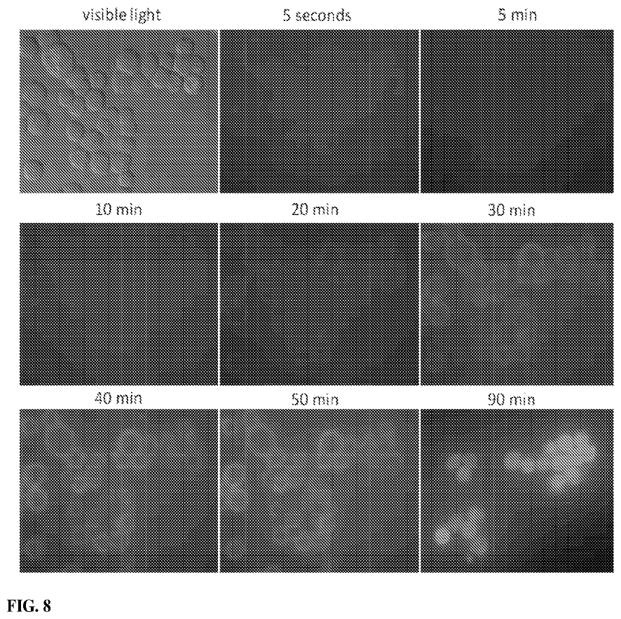

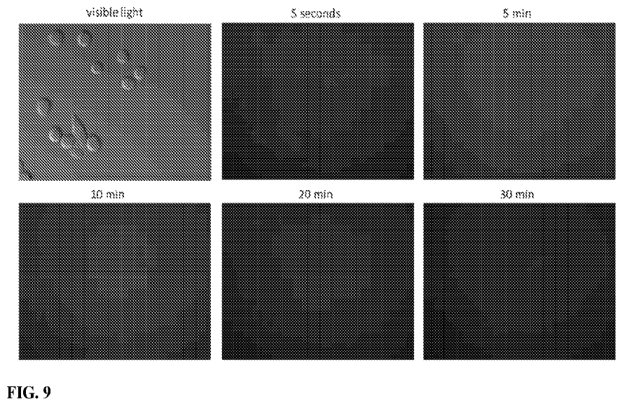

A study was conducted to determine cellular uptake of the CEACAM6-800 conjugate into the ZR-75.30 cell line with high cell-surface expression of CEACAM6 protein. A CEACAM6-800 was added to clear cell-culture medium and images acquired using an epi-fluorescence microscope equipped with an incubation chamber. Images were acquired over a 50-minute time course, and then again at 90 minutes using a different field of view. See FIG. 8. Uptake was observed at the periphery of cells as early as 5 seconds after adding CEACAM6-800. Uptake increased at the cell surface up to 50 minutes post-addition. By the 90-minute image acquisition, CEACAM6-800 had been internalized into the cells. A blocking study was performed using the same method except that a 10-fold excess of unlabeled CEACAM6 antibody fragment was added with CEACAM6-800. Live-cell uptake was significantly decreased following blocking. See FIG. 9.

### E. REFERENCES

Barkey N M, Preihs C, Cornnell H H, Martinez G, Carie A, Vagner J, Xu L, Lloyd M C, Lynch V M, Hruby V J, Sessler J L, Sill K N, Gillies R J, Morse D L. Development and in vivo quantitative magnetic resonance imaging of polymer micelles targeted to the melanocortin 1 receptor. Journal of medicinal chemistry. 2013; 56(16):6330-8. doi: 10.1021/jm4005576. PubMed PMID: 23863078; PubMed Central PMCID: PMCPMC3807968.
Barkey N M, Tafreshi N K, Josan J S, De Silva C R, Sill K N, Hruby V J, Gillies R J, Morse D L, Vagner J. Development of melanoma-targeted polymer micelles by conjugation of a melanocortin 1 receptor (MC1R) specific ligand. Journal of medicinal chemistry. 2011; 54(23):8078-84. doi: 10.1021/jm201226w. PubMed PMID: 22011200; PubMed Central PMCID: PMCPMC3302579.
Bartelink H, van Dam F, van Dongen J. Psychological effects of breast conserving therapy in comparison with radical mastectomy. Int J Radiat Oncol Biol Phys. 1985; 11(2):381-5.
Bathla L, Harris A, Davey M, Sharma P, Silva E. High resolution intra-operative two-dimensional specimen mammography and its impact on second operation for re-excision of positive margins at final pathology after breast conservation surgery. The American Journal of Surgery. 2011; 202(4):387-94. doi: 10.1016/j.amjsurg.2010.09.031.
Beauchemin N, Arabzadeh A. Carcinoembryonic antigen-related cell adhesion molecules (CEACAMs) in cancer progression and metastasis. Cancer Metastasis Rev. 201; 32(3-4):643-71. doi: 10.1007/s10555-013-9444-6. PubMed PMID: 23903773.
Blumenthal R D, Hansen H J, Goldenberg D M. Inhibition of adhesion, invasion, and metastasis by antibodies targeting CEACAM6 (NCA-90) and CEACAMS (Carcinoembryonic Antigen). Cancer Res. 2005; 65(19):8809-17. doi: 10.1158/0008-5472.CAN-05-0420. PubMed PMID: 16204051.
Cohen A S, Khalil F K, Welsh E A, Schabath M B, Enkemann S A, Davis A, Zhou J M, Boulware D C, Kim J, Haura E B, Morse D L. Cell-surface marker discovery for lung cancer. Oncotarget. 2017; 8(69):113373-402. doi: 10.18632/oncotarget.23009. PubMed PMID: 29371917; PubMed Central PMCID: PMCPMC5768334.
Cohen A S, Patek R, Enkemann S A, Johnson J O, Chen T, Toloza E, Vagner J, Morse D L. Delta-Opioid Receptor (deltaOR) Targeted Near-Infrared Fluorescent Agent for Imaging of Lung Cancer: Synthesis and Evaluation In Vitro and In Vivo. Bioconjug Chem. 2016; 27(2):427-38. doi: 10.1021/acs.bioconjchem.5b00516. PubMed PMID: 26488422; PubMed Central PMCID: PMCPMC5524213.
Coopey S, Buckley J, Smith B, Hughes K, Gadd M, Specht M. Lumpectomy Cavity Shaved Margins Do Not Impact Re-excision Rates in Breast Cancer Patients. Annals of Surgical Oncology. 2011; 18(11):3036-40. doi: 10.1245/s10434-011-1909-7.
Fisher B, Bauer M, Margolese R, Poisson R, Pilch Y, Redmond C, Fisher E, Wolmark N, Deutsch M, Montague E, Saffer E, Wickerham L, Lerner H, Glass A, Shibata H, Deckers P, Ketcham A, Oishi R, Russell I. Five-Year Results of a Randomized Clinical Trial Comparing Total Mastectomy and Segmental Mastectomy with or without Radiation in the Treatment of Breast Cancer. New England Journal of Medicine. 1985; 312(11):665-73. doi: doi:10.1056/NEJM198503143121101. PubMed PMID: 3883167.
Freedman G, Fowble B, Hanlon A, Nicolaou N, Fein D, Hoffman J, Sigurdson E, Boraas M, Goldstein L. Patients with early stage invasive cancer with close or positive margins treated with conservative surgery and radiation have an increased risk of breast recurrence that is delayed by adjuvant systemic therapy. Int J Radiat Oncol Biol Phys. 1999; 44(5):1005-15. Epub 1999/07/27. PubMed PMID: 10421533.
Gail M, Costantino J, Bryant J, Croyle R, Freedman L, Helzlsouer K, Vogel V. Weighing the risks and benefits of tamoxifen treatment for preventing breast cancer. J Natl Cancer Inst. 1999; 91(21):1829-46.
Hirche C, Murawa D, Mohr Z, Kneif S, Hunerbein M. ICG fluorescence-guided sentinel node biopsy for axillary nodal staging in breast cancer. Breast Cancer Res Treat. 2010; 121:373-8.
Huynh A S, Chung W J, Cho H I, Moberg V E, Celis E, Morse D L, Vagner J. Novel toll-like receptor 2 ligands for targeted pancreatic cancer imaging and immunotherapy. Journal of medicinal chemistry. 2012; 55(22):9751-62. Epub 2012/10/27. doi: 10.1021/jm301002f. PubMed PMID: 23098072; PubMed Central PMCID: PMC3861893.
Huynh A S, Estrella V, Stark V E, Cohen A S, Chen T, Casagni T J, Josan J S, Lloyd M C, Johnson J, Kim J, Hruby V J, Vagner J, Morse D L. Tumor Targeting and Pharmacokinetics of a Near-Infrared Fluorescent-Labeled delta-Opioid Receptor Antagonist Agent, Dmt-Tic-Cy5. Molecular pharmaceutics. 2016; 13(2):534-44. doi: 10.1021/acs.molpharmaceut.5b00760. PubMed PMID: 26713599; PubMed Central PMCID: PMCPMC4936951.
Kobbermann A, Unzeitig A, Xie X-J, Yan J, Euhus D, Peng Y, Sarode V, Moldrem A, Marilyn Leitch A, Andrews V, Stallings C, Rao R. Impact of Routine Cavity Shave Margins on Breast Cancer Re-excision Rates. Annals of Surgical Oncology. 2011; 18(5):1349-55. doi: 10.1245/s 10434-010-1420-6.
Korb M L, Hartman Y E, Kovar J, Zinn K R, Bland K I, Rosenthal E L. Use of monoclonal antibody-IRDye800C W bioconjugates in the resection of breast cancer. The Journal of surgical research. 2014; 188(1):119-28. Epub 2013/12/24. doi: 10.1016/j.jss.2013.11.1089. PubMed PMID: 24360117.
Lee M, Bhati R, von Rottenthaler E, Reagan A, Karver S, Reich R, Quinn G. Therapy choices and quality of life in young breast cancer survivors: a short-term follow-up. Am J Surg. 2013; 206(5):625-31.
McGuire K, Santillan A, Kaur P, Meade T, Parbhoo J, Mathias M, Shamehdi C, Davis M, Ramos D, Cox C. Are Mastectomies on the Rise? A 13-Year Trend Analysis of the Selection of Mastectomy Versus Breast Conservation Therapy in 5865 Patients. Annals of Surgical Oncology. 2009; 16(10):2682-90.
Millon S R, Ostrander J H, Yazdanfar S, Brown J Q, Bender J E, Rajeha A, Ramanujam N. Preferential accumulation of 5-aminolevulinic acid-induced protoporphyrin IX in breast cancer: a comprehensive study on six breast cell lines with varying phenotypes. Journal of biomedical optics. 2010; 15(1):018002. Epub 2010/03/10. doi: 10.1117/1.3302811. PubMed PMID: 20210488; PubMed Central PMCID: PMC2839804.
Moran M S, Schnitt S J, Giuliano A E, Harris J R, Khan S A, Horton J, Klimberg S, Chavez-MacGregor M, Freedman G, Houssami N, Johnson P L, Morrow M. Society of Surgical Oncology—American Society for Radiation Oncology Consensus Guideline on Margins for Breast-Conserving Surgery With Whole-Breast Irradiation in Stages I and II Invasive Breast Cancer. Annals of Surgical Oncology. 2014; 21(3):704-16. doi: 10.1245/s10434-014-3481-4.
Morrow M, White J, Moughan J, Owen J, Pajack T, Sylvester J, Frank Wilson J, Winchester D. Factors Predicting the Use of Breast-Conserving Therapy in Stage I and II Breast Carcinoma. Journal of Clinical Oncology. 2001; 19(8):2254-62.
Morse D L, Balagurunathan Y, Hostetter G, Trissal M, Tafreshi N K, Burke N, Lloyd M, Enkemann S, Coppola D, Hruby V J, Gillies R J, Han H. Identification of novel pancreatic adenocarcinoma cell-surface targets by gene expression profiling and tissue microarray. Biochem Pharmacol. 2010; 80(5):748-54. doi: 10.1016/j.bcp.2010.05.018. PubMed PMID: 20510208; PubMed Central PMCID: PMCPMC2914681.
Nagaya T, Nakamura Y A, Choyke P L, Kobayashi H. Fluorescence-Guided Surgery. Front Oncol. 2017; 7:314. doi: 10.3389/fonc.2017.00314. PubMed PMID: 29312886; PubMed Central PMCID: PMCPMC5743791.
NIH Consensus Statement: Treatment of early-stage breast cancer JAMA: The Journal of the American Medical Association. 1990; 265:668-70.
Olson T, Harter J, Mulloz A, Mahvi D, Breslin T M. Frozen Section Analysis for Intraoperative Margin Assessment During Breast-Conserving Surgery Results in Low Rates of Re-excision and Local Recurrence. Annals of Surgical Oncology. 2007; 14(10):2953-60. doi: 10.1245/s10434-007-9437-1.
Park C C, Mitsumori M, Nixon A, Recht A, Connolly J, Gelman R, Silver B, Hetelekidis S, Abner A, Harris J R, Schnitt S J. Outcome at 8 Years After Breast-Conserving Surgery and Radiation Therapy for Invasive Breast Cancer: Influence of Margin Status and Systemic Therapy on Local Recurrence. Journal of Clinical Oncology. 2000; 18(8):1668-75.
Pleijhuis R G, Graafland M, Vries Jd, Bart J, Jong J Sd, Dam G Mv. Obtaining adequate surgical margins in breast-conserving therapy for patients with early-stage breast cancer: current modalities and future directions. Ann Surg Oncol. 2009; 16:2717-30.
Polom K, Murawa D, Nowaczyk P, Rho Y, Murawa P. Breast cancer sentinel lymph node mapping using near infrared guided indocyanine green and indocyanine green-human serum albumin in comparison with gamma emitting radioactive colloid tracer. EJSO. 2012; 38:137-42.
Riley C J, Engelhardt K P, Saldanha J W, Qi W, Cooke L S, Zhu Y, Narayan S T, Shakalya K, Croce K D, Georgiev I G, Nagle R B, Garewal H, Von Hoff D D, Mahadevan D. Design and activity of a murine and humanized anti-CEACAM6 single-chain variable fragment in the treatment of pancreatic cancer. Cancer Res. 2009; 69(5):1933-40. doi: 10.1158/0008-5472.CAN-08-2707. PubMed PMID: 19244123; PubMed Central PMCID: PMCPMC2672909.
Rizeq B, Zakaria Z, Ouhtit A. Towards understanding the mechanisms of actions of carcinoembryonic antigen-related cell adhesion molecule 6 in cancer progression. Cancer Sci. 2018; 109(1):33-42. doi: 10.1111/cas.13437. PubMed PMID: 29110374; PubMed Central PMCID: PMCPMC5765285.
Sabel M S, Rogers K, Griffith K, Jagsi R, Kleer C G, Diehl K A, Breslin T M, Cimmino V M, Chang A E, Newman L A. Residual disease after re-excision lumpectomy for close margins. Journal of Surgical Oncology. 2009; 99(2):99-103. doi: 10.1002/jso.21215.
Sewda K, Coppola D, Enkemann S, Yue B, Kim J, Lopez A S, Wojtkowiak J W, Stark V E, Morse B, Shibata D, Vignesh S, Morse D L. Cell-surface markers for colon adenoma and adenocarcinoma. Oncotarget. 2016; 7(14):17773-89. doi: 10.18632/oncotarget.7402. PubMed PMID: 26894861; PubMed Central PMCID: PMCPMC4951249.
Tafreshi N K, Bui M M, Bishop K, Lloyd M C, Enkemann S A, Lopez A S, Abrahams D, Carter B W, Vagner J, Grobmyer S R, Gillies R J, Morse D L. Noninvasive detection of breast cancer lymph node metastasis using carbonic anhydrases IX and XII targeted imaging probes. Clin Cancer Res. 2012; 18(1):207-19. Epub 2011/10/22. doi: 10.1158/1078-0432.CCR-11-0238. PubMed PMID: 22016510.
Tafreshi N K, Enkemann S A, Bui M M, Lloyd M C, Abrahams D, Huynh A S, Kim J, Grobmyer S R, Carter W B, Vagner J, Gillies R J, Morse D L. A mammaglobin-A targeting agent for noninvasive detection of breast cancer metastasis in lymph nodes. Cancer Res. 2011; 71(3):1050-9. Epub 2010/12/21. doi: 10.1158/0008-5472.CAN-10-3091. PubMed PMID: 21169406.
Tafreshi N K, Huang X, Moberg V E, Barkey N M, Sondak V K, Tian H, Morse D L, Vagner J. Synthesis and characterization of a melanoma-targeted fluorescence imaging probe by conjugation of a melanocortin 1 receptor (MC1R) specific ligand. Bioconjug Chem. 2012; 23(12):2451-9. doi: 10.1021/bc300549s. PubMed PMID: 23116461; PubMed Central PMCID: PMCPMC3563353.
Tafreshi N K, Lloyd M C, Proemsey J B, Bui M M, Kim J, Gillies R J, Morse D L. Evaluation of CAIX and CAXII Expression in Breast Cancer at Varied O2 Levels: CAIX is the Superior Surrogate Imaging Biomarker of Tumor Hypoxia. Mol Imaging Biol. 2016; 18(2):219-31. doi: 10.1007/s11307-015-0885-x. PubMed PMID: 26276155; PubMed Central PMCID: PMCPMC4754166.
Tafreshi N K, Silva A, Estrella V C, McCardle T W, Chen T, Jeune-Smith Y, Lloyd M C, Enkemann S A, Smalley K S, Sondak V K, Vagner J, Morse D L. In vivo and in silico pharmacokinetics and biodistribution of a melanocortin receptor 1 targeted agent in preclinical models of melanoma. Molecular pharmaceutics. 2013; 10(8):3175-85. Epub 2013/06/15. doi: 10.1021/mp400222j. PubMed PMID: 23763620; PubMed Central PMCID: PMC3785103.
Terwisscha van Scheltinga A G, van Dam G M, Nagengast W B, Ntziachristos V, Hollema H, Herek J L, Schroder C P, Kosterink J G, Lub-de Hoog M N, de Vries E G. Intraoperative near-infrared fluorescence tumor imaging with vascular endothelial growth factor and human epidermal growth factor receptor 2 targeting antibodies. J Nucl Med. 2011; 52(11):1778-85. Epub 2011/10/13. doi: 10.2967/jnumed.111.092833. PubMed PMID: 21990576.
Troyan S L, Kiazad V, Gibbs-Strauss S L, Gioux S, Matsui A, Oketokoun R, Ngo L, Khamene A, Azar F, Fragioni J. The FLARE Intraoperative Near-Infrared Fluorescence Imaging System: A First-in-Human Clincal Trial in Breast Cancer Sentinel Lymph Node Mapping. Ann Surg Oncol. 2009; 16:2943-52.
Zavagno G, Dona M, Orvieto E, Mocellin S, Pasquali S, Goldin E, Lo Mele M, Belardinelli V, Nitti D. Separate cavity margins excision as a complement to conservative breast cancer surgery. European Journal of Surgical Oncology (EJSO). 2010; 36(7):632-8. doi: 10.1016/j.ejso.2010.05.018.

1. An anti-CEACAM6 imaging agent comprising the formula Y-R, wherein Y comprises an anti-CEACAM6 binding moiety and R comprises a first detectable label; wherein the first detectable label “R” comprises any dye that emits fluorescent light that is detectable on a clinical optical NIRF imager.

2. The anti-CEACAM6 imaging agent of claim 1, wherein the dye comprises a near infrared dye selected from the group consisting of IRdye800, AlexaFluor 790, ZW-800, Indocyanine Green, and SO456.

3. The anti-CEACAM6 imaging agent of claim 1, wherein the anti-CEACAM6 binding moiety comprises an antibody or anti-CEACAM6 binding antibody fragment.

4. The anti-CEACAM6 imaging agent of claim 3, wherein the anti-CEACAM6 binding moiety comprises an antibody fragment comprising an scFv, F(ab′)2, scFv-Fc (IgG4), or Fab′ that binds CEACAM6.

5. The anti-CEACAM6 imaging agent of claim 1, further comprising a second detectable label.

6. The anti-CEACAM6 imaging agent of claim 5, wherein the second label is a radiolabel.

7. The anti-CEACAM6 imaging agent of claim 1, wherein the anti-CEACAM6 binding moiety is conjugated to the detectable label via a NHS-ester linkage.

8. The anti-CEACAM6 imaging agent of claim 1, wherein the imaging agent is detectable using real-time molecular fluorescence imaging.

9. The anti-CEACAM6 imaging agent of claim 1 for use in imaging breast cancer tissue.

10. The anti-CEACAM6 imaging agent of claim 9, wherein the breast cancer tissue is marginal breast cancer tissue.

11. The anti-CEACAM6 imaging agent of claim 9, wherein the agent is administered for imaging marginal breast cancer tissue following lumpectomy.

12. The use of A method of performing surgical removal of a tumor in a subject comprising:
a) administering to a subject the anti-CEACAM6 imaging agent of claim 1; and
b) guiding the surgical removal of the tumor using optical surgical navigation.

13. The method of claim 12, wherein the method further comprises assaying the tumor using the anti-CEACAM6 imaging agent to determine the possibility of surgical removal after step a and prior to step b.

14. The method of claim 12, wherein the method further comprises monitoring the progress of the surgical removal.

15. The method of claim 12, wherein the anti-CEACAM6 imaging agent comprises a second detectable label and wherein the second label is a radiolabel.

16. The method of claim 12 wherein the method is performed following lumpectomy.

17. The method of claim 12 wherein the method is performed following systemic administration for intraoperative margin assessment.
